(19) United States
(12) Patent Application Publication
(10) Pub. No.: US 2013/0022975 A1
(43) Pub. Date: Jan. 24, 2013

(54) METHOD FOR DETECTING ARTERIOSCLEROTIC DISEASES ON THE BASIS OF SINGLE NUCLEOTIDE POLYMORPHISM AT HUMAN CHROMOSOME 5P15.3

(71) Applicants: Kouichi Ozaki, Yokohama-shi (JP); Toshihiro Tanaka, Yokohama-shi (JP); Yusuke Nakamura, Yokohama-shi (JP)

(72) Inventors: Kouichi Ozaki, Yokohama-shi (JP); Toshihiro Tanaka, Yokohama-shi (JP); Yusuke Nakamura, Yokohama-shi (JP)

(73) Assignee: RIKEN, Saitama (JP)

(21) Appl. No.: 13/579,463

(22) PCT Filed: Feb. 18, 2011
(86) PCT No.: PCT/JP2011/053542

§ 371 Date: Sep. 27, 2012

(30) Foreign Application Priority Data
Feb. 18, 2010 (JP) 2010-033913

(51) Int. Cl. C12Q 1/68; C07H 21/04

(52) U.S. Cl. 435/6.11; 536/24.31; 536/24.33

(57) ABSTRACT

An atherosclerotic disease such as myocardial infarction or angina pectoris is detected by analyzing a single nucleotide polymorphism on human chromosome 5p15.3, and by associating results of the analysis with the risk of the onset thereof. Examples of the single nucleotide polymorphism on human chromosome 5p15.3 include a nucleotide corresponding to the nucleotide at position 61 in the nucleotide sequence of SEQ ID NO: 1, SEQ ID NO: 2, or SEQ ID NO: 3, and a polymorphism at a nucleotide which is in linkage disequilibrium with the above nucleotide.

### TECHNICAL FIELD

The present invention relates to a method of detecting a risk of an arteriosclerotic disease such as myocardial infarction, angina pectoris or the like, and a reagent used therefor.

### BACKGROUND ART

In many developed countries with an European or American lifestyle, arteriosclerotic diseases such as coronary artery diseases including myocardial infarction (MI), or the like is a primary cause of the fatality and morbidity among delayed diseases (Non-patent Documents 1 and 2). When it comes to the onset of myocardial infarction, there are often cases where a spate of severe complications (in particular, ventricular fibrillation and cardiac rupture which may cause sudden death) occurs without any preceding clinical signs. Although recent progress in diagnosis and treatment for myocardial infarction has drastically improved quality in treatment and diagnosis for myocardial infarction, the morbidity rate of myocardial infarction remains high.

Epidemiological studies have identified a variety of risk factors for arteriosclerotic diseases (type 2 diabetes mellitus, hypercholesterolemia, hypertension, obesity, and the like). There are also some reports on genetic factors for myocardial infarction. For example, it has been reported that a risk for myocardial infarction is 2 to 7 times higher in a family member related in the first degree of kinship to a patient who developed acute myocardial infarction before the age of 55 years (Non-patent Document 3). In addition, a study of twins has demonstrated that, when one of twins died of myocardial infarction before the age of 55 years, the other has an eight-fold increased risk of death from myocardial infarction (Non-patent Document 4).

Moreover, thus far, by case-control studies for examining an association of linkage analysis or single nucleotide polymorphisms (SNP), several genetic variants which enhance myocardial infarction susceptibility have been identified in several genomic loci (Patent Documents 1 to 8 and Non-patent Documents 5 to 11).

### PRIOR ART REFERENCES

### Patent Documents

Patent Document 1: Japanese Patent Application Laid-Open Publication No. 2008-072947
Patent Document 2: Japanese Patent Application Laid-Open Publication No. 2008-048627
Patent Document 3: Japanese Patent Application Laid-Open Publication No. 2007-330166
Patent Document 4: Japanese Domestic Re-Publication of PCT International Application No. 2006/075626
Patent Document 5: Japanese Domestic Re-Publication of PCT International Application No. 2006/073183
Patent Document 6: Japanese Domestic Re-Publication of PCT International Application No. 2006/068239
Patent Document 7: Japanese Domestic Re-Publication of PCT International Application No. 2005/017200
Patent Document 8: Japanese Domestic Re-Publication of PCT International Application No. 2004/015100

### Non-Patent Documents

Non-patent Document 1: Breslow J. W. (1997) Nat. Med. 3; 600-601
Non-patent Document 2: Braunwald E (1997) N. Engl. J. Med. 337:1360-136
Non-patent Document 3: Lusis A J et al. (2004) Annu Rev Genomics Hum Genet. 5:189-218
Non-patent Document 4: Marenberg M E et al. (1994) N Engl J Med 330: 1041-1046
Non-patent Document 5: Topol E J et al. (2001) Circulation 104:2641-2644
Non-patent Document 6: Yamada Y et al. (2002) N. Engl. J. Med. 347: 1916-1923
Non-patent Document 7: Ozaki K et al. (2002) Nat. Genet. 32: 650-654
Non-patent Document 8: Ozaki K et al. (2004) Nature 429: 72-75
Non-patent Document 9: Ozaki K et al. (2006) Nat. Genet. 38: 921-925
Non-patent Document 10: Stenina O I et al. (2003) Circulation 108 (12): 1514-1519
Non-patent Document 11: Helgadottir A et al (2004) Nat. Genet. 36:233-239

### SUMMARY OF THE INVENTION

### Problems to be Solved by the Invention

An object of the present invention is to provide a detection method for predicting with high accuracy a risk of developing an arteriosclerotic disease such as myocardial infarction, angina pectoris or the like, and a detection reagent used therefor.

In order to solve the above-mentioned problem, the present inventors intensively studied to discover that single nucleotide polymorphisms (SNPs) present on the short arm of human chromosome 5 region p15.3 are associated with the onset of an arteriosclerotic disease such as myocardial infarction, angina pectoris, or the like. They found that the susceptibility of developing arteriosclerotic disease such as myocardial infarction, angina pectoris, or the like can be accurately estimated by examining these polymorphisms, thereby completed the present invention.

Accordingly, the present invention is as follows:

(1) A method of detecting an arteriosclerotic disease comprising the steps of analyzing a single nucleotide polymorphism present on a human chromosome 5p15.3 region and associating a result of the analysis with a risk of developing said arteriosclerotic disease.

(2) The method according to (1), wherein said single nucleotide polymorphism is a polymorphism of a nucleotide corresponding to the nucleotide at position 61 in a nucleotide sequence of SEQ ID NO: 1, SEQ ID NO: 2, or SEQ ID NO: 3, or a nucleotide in linkage disequilibrium with said nucleotide.

(3) The method according to (1) or (2), wherein said arteriosclerotic disease is a coronary artery disease.

(4) The method according to (3), wherein said coronary artery disease is cardiac infarction or angina pectoris.

(5) A probe for detecting an arteriosclerotic disease, said probe comprising a sequence of 10 or more nucleotides including the nucleotide at position 61 in SEQ ID NO: 1, SEQ ID NO: 2, or SEQ ID NO: 3, or a complementary sequence thereof.

(6) A primer for detecting an arteriosclerotic disease, said primer being capable of amplifying a region including the nucleotide at position 61 in the nucleotide sequence of SEQ ID NO: 1, SEQ ID NO: 2, or SEQ ID NO: 3.

### BRIEF DESCRIPTION OF THE DRAWINGS

FIG. 1 is a FIGURE showing a location of rs11748327 on human chromosome 5p15.3 and a linkage disequilibrium (LD) block in the vicinity thereof.

### EMBODIMENTS FOR CARRYING OUT THE INVENTION

### <1> Detection Method of the Present Invention

A detection method of the present invention is a method of analyzing a single nucleotide polymorphism on a region of the short arm of human chromosome 5, p15.3, and examining the onset of the arteriosclerotic disease based on results of the analysis (whether or not such a nucleotide is a disease susceptibility allele). Examples of the arteriosclerotic disease include an arteriosclerotic disease in an artery of the brain such as cerebral infarction, cerebral hemorrhage, or the like; an arteriosclerotic disease in a coronary artery (coronary artery disease: CAD) such as myocardial infarction, angina pectoris, or the like; an arteriosclerotic disease in the aorta such as aortic aneurysm, aortic dissection, or the like; an arteriosclerotic disease in a renal artery such as nephrosclerosis and renal failure caused thereby, or the like; and an arteriosclerotic disease in a peripheral artery such as arteriosclerosis obliterans or the like. Results of the analysis of the single nucleotide polymorphism are associated with the risk of developing the arteriosclerotic disease.

A human chromosome 5p15.3 region includes, for example, a region approximately from 3900000 to 4100000 of GenBank Accession No. NT_{—}006576.15. Because there may possibly be substitutions or deletions at nucleotides other than nucleotides associated with arteriosclerotic diseases in a nucleotide sequence on the human chromosome 5p15.3 region owing to racial difference or the like, the region is not limited to the above sequence.

Examples of a single nucleotide polymorphism on the human chromosome 5p15.3 region associated with an arteriosclerotic disease include rs11748327, rs490556, rs521660, and the like. This rs number represents a registration number in the dbSNP database of the National Center for Biotechnology Information (//www.ncbi.nlm.nih.gov/projects/SNP/).

rs11748327 refers to a cytosine (C)/thymine (T) polymorphism at the nucleotide at position 4019789 in GenBank Accession No. NT_{—}006576.15 and a high risk of developing an arteriosclerotic disease is indicated when this nucleotide is C. When an analysis is carried out by taking an allele into consideration, the allele indicating the risk of developing the arteriosclerotic disease in order from the highest to the lowest is: CC, CT, and TT.

rs490556 refers to a thymine (T)/cytosine (C) polymorphism at the nucleotide at position 4012650 in GenBank Accession No. NT_{—}006576.15 and a high risk of developing an arteriosclerotic disease is indicated when this nucleotide is T. When an analysis is carried out by taking an allele into consideration, the allele indicating the risk of developing the arteriosclerotic disease in order from the highest to the lowest is: TT, TC, and CC.

rs521660 refers to a guanine (G)/adenine (A) polymorphism at the nucleotide at position 4025932 in GenBank Accession No. NT_{—}006576.15 and a high risk of developing an arteriosclerotic disease is indicated when this nucleotide is G. When an analysis is carried out by taking an allele into consideration, the allele indicating the risk of developing the arteriosclerotic disease in order from the highest to the lowest is: GG, GA, and AA.

For rs11748327, rs490556, and rs521660, a sequence of total 121 bp in length which covers the SNP nucleotide and 60-bp regions upstream and downstream therefrom are shown SEQ ID NOs: 1, 2, and 3, respectively. Each has a polymorphism of the nucleotide at position 61.

Nucleotides corresponding to these nucleotides are analyzed in the present invention. The phrase “corresponding to” herein means a corresponding nucleotide in a region including the above sequence on the human chromosome 5p15.3 region; and even if positions other than SNPs in the above sequence slightly varies because of racial difference or the like, analysis of the corresponding nucleotide therein is included.

An arteriosclerotic disease can be detected by examining the kind of the nucleotide of the above SNP, and associating with the arteriosclerotic disease based on the index as described above. The number of the SNP to be examined may be one; or it may be two or more (haplotype analysis). The nucleotide sequence of the sense strand may be analyzed or that of the antisense strand may be analyzed. For example, in the case of rs11748327, when the antisense strand thereof is analyzed, it turns out to be a G/A polymorphism and G is a disease susceptibility allele.

In addition, a nucleotide to be analyzed in the present invention is not limited to the above. A polymorphism of a nucleotide in linkage disequilibrium with the above nucleotide may be analyzed. The phrase “a nucleotide in linkage disequilibrium with the above nucleotide” herein refers to a nucleotide which satisfies a relation of r²>0.5, preferably r²>0.8 with the above nucleotide. A concrete example includes one shown in Table 1. These sequences and the kind of polymorphism can be referred to the above dbSNP database.

TABLE 1
Coronary artery disease associated SNPs in 5p15.3

| Significant SNPs | SNPs that are in linkage disequilibrium with the significant SNP | | | | | | |
|---|---|---|---|---|---|---|---|
| rs521660 | rs2452877 | rs1187484 | rs554079 | rs613885 | rs13157330 | rs578880 | rs12513797 |
| | rs555411 | rs10076234 | rs2459732 | rs1911909 | rs9313087 | rs527841 | rs2388656 |
| | rs2136174 | rs10462783 | rs825724 | rs534161 | rs2452878 | rs4432895 | rs1187475 |
| | rs1187488 | rs1703291 | rs494074 | rs1187463 | rs1187471 | rs484410 | rs1201368 |
| rs11748327 | rs17718387 | rs17719935 | rs11739657 | rs12659815 | rs1353102 | rs16872786 | rs10041378 |
| | rs8895675 | rs11134030 | rs9313069 | rs11743330 | rs1911908 | rs2047074 | rs17662489 |
| rs490556 | rs4702313 | rs660733 | rs937219 | rs493478 | rs13178917 | rs903085 | rs13190544 |
| | rs3943033 | rs645402 | rs11738089 | rs2018939 | | | |

| Significant SNPs | SNPs that are in linkage disequilibrium with the significant SNP | | | | | total SNP No. |
|---|---|---|---|---|---|---|
| rs521660 | rs2459731 | rs511907 | rs483632 | rs2459733 | rs4490587 | 47 |
| | rs1108724 | rs1703278 | rs1493463 | rs493049 | rs4702376 | |
| | rs580841 | rs1703290 | rs1493464 | rs1187474 | rs545066 | |
| | rs1703285 | rs493452 | rs1105521 | | | |
| rs11748327 | rs2398655 | rs16872792 | rs7706181 | rs6864370 | rs1493470 | 24 |
| | rs10039404 | rs10512704 | rs17717616 | rs11750208 | | |
| rs490556 | rs1878569 | rs10071096 | rs500818 | rs540937 | rs10060853 | 17 |

A sample to be used for analysis for genetic polymorphisms on human chromosome 5p15.3 is not particularly restricted as long as it is a sample including chromosomal DNA. Examples thereof include a body fluid sample such as blood, urine or the like, cells such as liver cells or the like, body hair such as hair or the like. These samples can be directly used for the analysis of genetic polymorphisms but it is preferred that the chromosomal DNA be isolated from these samples by a conventional method and then used for the analysis.

Analysis of genetic polymorphisms on human chromosome 5p15.3 can be carried out by a usual method of analyzing the gene polymorphisms. Examples thereof include sequence analysis, PCR, hybridization, and the like but are not limited thereto.

Sequencing can be carried out by a usual method. Specifically, a sequence reaction is carried out using primers located several dozen nucleotides in the 5′ side of a polymorphic nucleotide and, from the results of the analysis, the kind of nucleotide at the corresponding position is determined.

An analysis can also be carried out by examining the presence or absence of amplification by PCR. For example, primers having a sequence corresponding to a region including a polymorphic nucleotide as well as corresponding to the respective polymorphism are individually prepared. PCR is carried out using each primer and the kind of polymorphism can be determined on the basis of the presence or absence of an amplified product.

Alternatively, the presence or absence of amplification can be examined using the LAMP method (Japanese Patent No. 3313358), the NASBA method (Nucleic Acid Sequence-Based Amplification; Japanese Patent No. 2843586), the ICAN method (Japanese Patent Application Laid-Open Publication No. 2002-233379), or the like. Besides, a single chain amplification method may be employed.

Further, a DNA fragment including a polymorphism may be amplified and the kind of polymorphism can also be determined by mobility difference in electrophoresis of the amplified product. An example of such a method includes the PCR-SSCP (single-strand conformation polymorphism) method (Genomics. 1992 Jan. 1; 12(1): 139-146). Specifically, DNA including a polymorphic site on human chromosome 5p15.3 is first amplified and the amplified DNA is then dissociated into single stranded DNAs. Subsequently, the dissociated single strand DNAs are separated on a non-denatured gel and the kind of polymorphism is determined based on difference in the mobility of the separated single strand DNAs on the gel.

Furthermore, in cases where a polymorphic nucleotide is included in a restriction enzyme recognition sequence, an analysis can be carried out on the basis of the presence or absence of cleavage by a restriction enzyme (the RFLP method). In this case, a DNA sample is first digested by a restriction enzyme. The DNA fragment is then separated and the kind of polymorphism is determined based on the size of detected DNA fragment.

The kind of polymorphism can also be analyzed by examining the presence or absence of hybridization. That is, probes corresponding to respective nucleotide are prepared and by examining which probe hybridizes to the resulting restriction fragment, the kind of SNP nucleotide can also be examined. By determining the kind of SNP nucleotide as described above, data for detecting an arteriosclerotic disease can be obtained.

### <2> Detection Reagent of the Present Invention

The present invention also provides a detection reagent such as a primer, a probe or the like for detecting the risk for developing an arteriosclerotic disease. An example of such a probe includes a probe including the above polymorphic site on human chromosome 5p15.3 and capable of accessing the kind of a nucleotide of the polymorphic site on the basis of the presence or absence of hybridization. Concrete examples thereof include a probe comprising a sequence of 10 or more nucleotides including the nucleotide at position 61 in SEQ ID NO: 1, 2, or 3, or a complementary sequence thereof. The length of probe is more preferably 15 to 35 nucleotides and still more preferably 20 to 35 nucleotides.

In addition, an example of the primer includes a primer which can be used in PCR for amplifying the above polymorphic site on human chromosome 5p15.3 or a primer which can be used for a sequence analysis (sequencing) of the above polymorphic site. A concrete example includes a primer capable of amplifying or sequencing a region including the nucleotide at position 61 in a nucleotide sequence of SEQ ID NO: 1, 2, or 3. The length of such a primer is preferably 10 to 50 nucleotides, more preferably 15 to 35 nucleotides, and still further preferably 20 to 35 nucleotides.

Examples of the primer for sequencing the above polymorphic site include a primer having a sequence of the 5′ side region, preferably 30 to 100 nucleotides upstream, of the above nucleotide; and a primer having a sequence complementary to the 3′ side region, preferably 30 to 100 nucleotides downstream, of the above nucleotide. Examples of a primer for accessing a polymorphism on the basis of the presence or absence of amplification by PCR include a primer which has a sequence including the above nucleotide and includes the above nucleotide in its 3′ terminus side, a primer which has a complementary sequence of a sequence including the above nucleotide and includes a complementary nucleotide of the above nucleotide in its 3′ terminus side, and the like.

The detection reagent of the present invention may include, in addition to those primers and probes, polymerase and buffers for PCR, reagents for hybridization, or the like.

### EXAMPLES

By way of examples, the present invention will be further concretely described below. However, the present invention is by no means limited thereto.

### Example 1

### Large Scale SNP Study for Examining Association with Myocardial Infarction (MI)

### (1) Materials and Method

### (1-1) DNA Sample

For a genome wide correlation analysis and following second analysis, samples of myocardial infarction patients and control subjects registered in the BioBank Japan project (//biobankjp.org/) were used. Characteristics of the third and fourth populations were same as described in Nat Genet. 38, 921-925 (2006) and Nat Genet. 41, 329-333 (2009) except that additional 1235 samples purchased from Health Science Research Resource Bank were used as control samples.

Myocardial infarction patients subjected to be analyzed were patients who had been diagnosed as myocardial infarction by satisfying two or more of three conditions (Nat Genet. 32 (4):650-4. 2002): (i) having a medical history of feeling of chest pressure, pain, tightness in the chest, or the like for 30 minutes or longer, (ii) showing 0.1 mV or larger of ST segment elevation in at least one standard lead or two precordial leads, and (iii) showing a more than higher concentration of serum creatine kinase than a standard value (Nat Genet. 32(4):650-4. 2002).

Samples of angina pectoris were also ones registered in the BioBank Japan. Angina pectoris was diagnosed in accordance with a standard described in J Am Coll Cardiol 36, 970-1062 (2000).

All of the subjects were Japanese. A consent document for taking part in this study was submitted by each of the subjects or a parent of the subject (when the subject is under 20 years of age), in accordance with a procedure approved by the ethical review committee of the Center for Genomic Medicine, Independent Administrative Institution, The Institute of Physical and Chemical Research (RIKEN).

### (1-2) Discovery of SNP and Genotype Analysis

A genome wide correlation analysis and a genotyping method of the second screening were carried out in accordance with a method described in Nat Genet. 40(9):1098-1102 (2008). In the third and fourth screenings, the genotyping was carried out by a multiplex-PCR invader assay which is described in J Hum Genet 46:471-477 (2001) and Nat Genet. 32 (4):650-654. (2002).

### (1-3) Statistical Analysis

A haplotype block and a haplotype frequency were analyzed in accordance with Haploview v4.0 (Bioinfomatics 21, 263-265 (2005)). A tag SNP was then selected in a pair wise tagging mode using Haploview software (Bioinfomatics 21, 263-265 (2005)) and applied to a permutation test in the haplotype analysis.

Also, the haplotype analysis was carried out using a THESIAS program (Tregouet et al. 2007) and a conditional log likelihood with Akaike's Information Criterion (AIC): AIC=−2×(the maximum value of the conditional log likelihood)+2×(the number of the parameters). As the number of the parameters, the number of the alleles/haplotypes which was used in each model and whose frequency is more than 0.01 was employed. In a logistic regression analysis of SNP, a likelihood-ratio test with degree of freedom of 1 (1-d.f.) was first carried out (Bioinfomatics 23(8), 1038-1039 (2007)) to determine which is more appropriate, a multiplicative allelic effect model with degree of freedom of 1 (1-d.f.) or a full genotype model with degree of freedom of 2 (2-d.f.). Because a significant difference (P>0.05) was not seen from the full genotype model, we postulated the multiplicative allelic effect model. Subsequently, a forward logistic regression analysis was carried out and, at first, whether the most significant SNP is sufficient as a model for association among SNP sets was analyzed. For this, on the premise of a synergistic allele effect to additional SNPs, in order to add the remaining each of the SNPs to the model, a (1-d.f.) likelihood test with degree of freedom of 1 was employed. An association between patient's clinical profile and genotype information was evaluated using a one-way ANOVA and a χ² test.

### (2) Results

### <Genome Wide Correlation Analysis>

First, 268,068 SNPs for 194 myocardial infarction patients (Cases) registered in the BioBank Japan and 1,539 control subjects (Controls) were analyzed (genome wide correlation analysis: primary analysis). As a result, genotype information for 210,785 SNPs was obtained. Among them, the second screening was carried out for 8,740 SNPs with P<0.02.

In the second screening, 1,394 myocardial infarction patients and 1,388 control subjects were analyzed and data for 7,374 SNPs could be obtained. Among them, two SNPs which showed statistical significance (cut-off P<0.0000068) after Bonferroni correction were identified. One of them was an SNP reported previously in Nat Genet. 41, 329-333 (2009). The other was SNP (rs11748327) on chromosome 5p15.3 showing P=1.8×10⁻⁶. As for this SNP, the third (1,500 myocardial infarction patients, 1,356 controls) and the fourth (2,283 myocardial infarction patients, 3,439 controls) screenings were carried out.

The results are shown in Table 2.

TABLE 2
Association of rs11748327 SNP with MI

| | | Cases | | | | | | | Controls | | | | | | |
|---|---|---|---|---|---|---|---|---|---|---|---|---|---|---|---|
| db SNP ID | Stage | 11 | (%) | 12 | (%) | 22 | (%) | SUM | 11 | (%) | 12 | (%) | 22 | (%) | SUM |
| rs11748327 | 1st | 130 | 67.0 | 52 | 26.8 | 12 | 6.2 | 194 | 866 | 56.3 | 575 | 37.4 | 97 | 6.3 | 1538 |
| C > T | 2nd | 896 | 64.4 | 436 | 31.3 | 60 | 4.3 | 1392 | 758 | 55.3 | 531 | 38.7 | 82 | 6.0 | 1371 |
| | 3rd | 938 | 62.8 | 480 | 32.1 | 76 | 5.1 | 1494 | 770 | 57.1 | 490 | 36.4 | 88 | 6.5 | 1348 |
| | 4th | 1437 | 62.9 | 740 | 32.4 | 106 | 4.6 | 2283 | 1964 | 57.5 | 1254 | 36.7 | 197 | 5.8 | 3415 |
| | combined | 3401 | 63.4 | 1708 | 31.8 | 254 | 4.7 | 5363 | 4358 | 56.8 | 2850 | 37.1 | 464 | 6.0 | 7672 |

| | | Comparison of allele frequency | | | |
|---|---|---|---|---|---|
| db SNP ID | Stage | χ² | P value | OR | 95% CI |
| rs11748327 | 1st | 5.5 | 1.9 × 10⁻² | 1.37 | 1.05-1.78 |
| C > T | 2nd | 22.8 | 1.8 × 10⁻⁶ | 1.36 | 1.20-1.54 |
| | 3rd | 10.2 | 1.4 × 10⁻³ | 1.22 | 1.08-1.38 |
| | 4th | 16.7 | 4.4 × 10⁻⁵ | 1.21 | 1.10-1.32 |
| | combined | 56.0 | 5.3 × 10⁻¹³* | 1.25 | 1.18-1.33 |

*P value was calculated by Mantel-Haenszel test

The data from all of the stages were combined using the Mantel-Haenszel test to obtain χ²=56.0 (P=5.3×10⁻¹³) and an odds ratio of 1.25 (95% confidence interval (CI) 1.18-1.33). It was thus found that this SNP showed a strong correlation with myocardial infarction.

### <Linkage Disequilibrium Analysis>

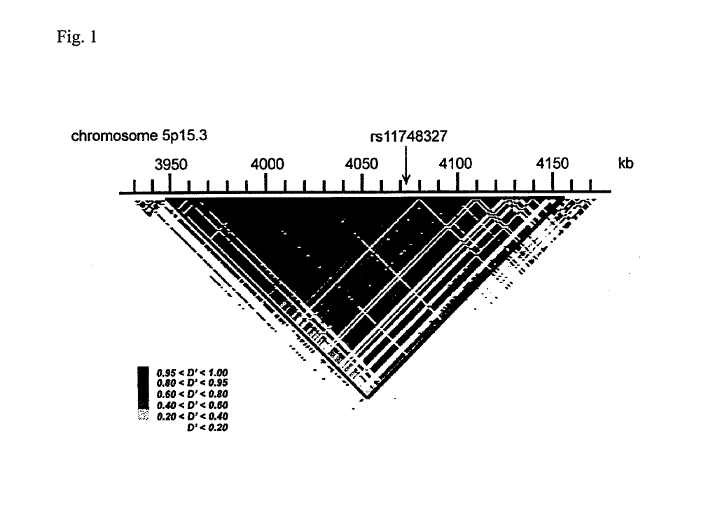

According to HapMap JPT data (http://www.hapmap.org; The International HapMap Consortium 2005), rs11748327 is located in a linkage disequilibrium (LD) block of about 250 kb which composed of SNPs with a minor allele frequency being not less than 20% (FIG. 1). In order to examine if other SNPs which associate with myocardial infarction exist in this block, in addition to rs11748327, 15 SNPs with a minor allele frequency being above 5% and a threshold value of r² being 0.8 were selected. When these SNPs were analyzed using samples of the above third screening, rs490556 and rs521660 were found to show significant correlation with myocardial infarction after Bonferroni correction (Table 3).

TABLE 3
Association analysis of the fifteen tag SNPs with MI

| | cases | | | | | | | Controls | | | | | | | | r² with |
|---|---|---|---|---|---|---|---|---|---|---|---|---|---|---|---|---|
| dbSNP ID | 11 | (%) | 12 | (%) | 22 | (%) | SUM | 11 | (%) | 12 | (%) | 22 | (%) | SUM | P value* | rs11748327 |
| rs511664 C > T | 1232 | 86.0 | 193 | 13.5 | 8 | 0.6 | 1433 | 1150 | 88.3 | 143 | 11.0 | 9 | 0.7 | 1302 | 1.6 × 10⁻¹ | 0.03 |
| rs505800 A > G | 763 | 51.4 | 599 | 40.3 | 123 | 8.3 | 1485 | 666 | 49.6 | 551 | 41.1 | 125 | 9.3 | 1342 | 4.0 × 10⁻¹ | 0.3 |
| rs2008927 C > T | 700 | 47.3 | 614 | 41.5 | 166 | 11.2 | 1480 | 650 | 49.2 | 524 | 39.6 | 148 | 11.2 | 1322 | 7.2 × 10⁻¹ | 0.18 |
| rs631942 G > A | 795 | 53.9 | 558 | 37.8 | 122 | 8.3 | 1475 | 660 | 48.9 | 569 | 42.1 | 121 | 9.0 | 1350 | 2.9 × 10⁻¹ | 0.4 |
| rs10060583 C > T | 566 | 38.1 | 686 | 46.2 | 233 | 15.7 | 1485 | 547 | 40.9 | 611 | 45.6 | 181 | 13.5 | 1339 | 9.1 × 10⁻¹ | 0.12 |
| rs490556 T > C | 863 | 58.1 | 521 | 35.1 | 102 | 6.9 | 1486 | 681 | 50.7 | 549 | 40.9 | 113 | 8.4 | 1343 | 2.4 × 10⁻³ | 0.59 |
| rs521660 G > A | 762 | 55.3 | 515 | 37.3 | 102 | 7.4 | 1379 | 627 | 49.1 | 530 | 41.5 | 120 | 9.4 | 1277 | 1.5 × 10⁻² | 0.79 |
| rs1187466 T > A | 630 | 44.3 | 630 | 44.3 | 162 | 11.4 | 1422 | 579 | 46.9 | 540 | 43.8 | 115 | 9.3 | 1234 | 1.1 × 10⁻¹ | 0.11 |
| rs1187463 C > A | 743 | 50.4 | 600 | 40.7 | 130 | 8.8 | 1473 | 607 | 46.0 | 563 | 42.7 | 150 | 11.4 | 1320 | 7.5 × 10⁻² | 0.65 |
| rs903083 C > T | 1334 | 89.4 | 156 | 10.5 | 2 | 0.1 | 1492 | 1180 | 88.0 | 155 | 11.6 | 6 | 0.4 | 1341 | 2.6 × 10⁻¹ | 0.02 |
| rs10512709 G > A | 607 | 41.3 | 682 | 46.5 | 179 | 12.2 | 1468 | 559 | 42.3 | 611 | 46.3 | 151 | 11.4 | 1321 | 8.0 × 10⁻¹ | 0.17 |
| rs1187477 C > T | 1169 | 77.9 | 308 | 20.5 | 23 | 1.5 | 1500 | 1025 | 75.6 | 311 | 22.9 | 20 | 1.5 | 1356 | 3.0 × 10⁻¹ | 0.04 |
| rs1209069 C > T | 816 | 58.6 | 496 | 35.6 | 81 | 5.8 | 1393 | 779 | 61.2 | 435 | 34.2 | 58 | 4.6 | 1272 | 1.4 × 10⁻¹ | 0.04 |
| rs1493470 G > A | 827 | 55.9 | 566 | 38.3 | 86 | 5.8 | 1479 | 729 | 54.5 | 504 | 37.7 | 104 | 7.8 | 1337 | 2.4 × 10⁻¹ | 0.78 |
| rs1187483 T > C | 578 | 39.6 | 663 | 45.4 | 220 | 15.1 | 1461 | 478 | 36.0 | 635 | 47.8 | 215 | 16.2 | 1328 | 1.2 × 10⁻¹ | 0.41 |

*Comparison of allelic frequency and adjusted for Bonferroni's correction.

rs490556 and rs521660 were in linkage disequilibrium with rs11748327 with r²=0.59 and 0.79, respectively.

When these SNPs were further analyzed using samples of the above the fourth screening, it was confirmed that these SNPs showed significant correlation with myocardial infarction (Table 4).

TABLE 4
Association of the three SNPs with CAD

| | | Cases | | | Controls | | | Comparison of allele frequency | | | |
|---|---|---|---|---|---|---|---|---|---|---|---|
| dbSNP ID | Samples | 11 | 12 | 22 | 11 | 12 | 22 | χ² | P value | OR | 95% CI |
| rs490556 | MI 3rd | 863 | 521 | 102 | 681 | 549 | 113 | 14.4 | 1.5 × 10⁻⁴ | 1.26 | 1.12-1.42 |
| T > C | MI 4th | 1324 | 802 | 141 | 1786 | 1399 | 254 | 20.6 | 5.7 × 10⁻⁶ | 1.22 | 1.12-1.33 |
| | combind | 2187 | 1323 | 243 | 2467 | 1948 | 367 | 33.8 | 4.0 × 10⁻⁹* | 1.23 | 1.15-1.32 |
| | UA vs combind CO | 1563 | 1015 | 187 | | | | 14.8 | 1.1 × 10⁻⁴ | 1.16 | 1.08-1.25 |
| rs11748327 | MI 3rd | 938 | 480 | 76 | 770 | 490 | 88 | 10.1 | 1.4 × 10⁻³ | 1.22 | 1.08-1.38 |
| C > T | MI 4th | 1437 | 740 | 106 | 1964 | 1254 | 197 | 16.7 | 4.4 × 10⁻⁵ | 1.21 | 1.10-1.30 |
| | combind | 2375 | 1220 | 182 | 2734 | 1744 | 285 | 26.4 | 2.5 × 10⁻⁷* | 1.21 | 1.13-1.31 |
| | UA vs combind CO | 1724 | 920 | 129 | | | | 17.2 | 3.4 × 10⁻⁵ | 1.18 | 1.09-1.28 |
| rs521660 | MI 3rd | 762 | 515 | 102 | 627 | 530 | 120 | 10.9 | 9.4 × 10⁻⁴ | 1.22 | 1.09-1.38 |
| G > A | MI 4th | 1197 | 781 | 162 | 1597 | 1342 | 299 | 21.8 | 3.1 × 10⁻⁶ | 1.23 | 1.13-1.34 |
| | combind | 1959 | 1296 | 264 | 2224 | 1872 | 419 | 32.7 | 1.2 × 10⁻⁸* | 1.23 | 1.14-1.32 |
| | UA vs combind CO | 1413 | 989 | 205 | | | | 15.4 | 8.7 × 10⁻⁵ | 1.16 | 1.08-1.25 |

*P values were calculated by Mantel-Haenszel test.

### <Haplotype Analysis>

An association between the haplotype of these three SNPs and myocardial infarction was examined by THESIAS (Bioinfomatics 23(8), 1038-1039 (2007)). As a result, it was found that haplotypes with the highest and the second highest frequency showed a strong association with myocardial infarction (Table 5).

TABLE 5
Haplotype analysis

| | | | | | | Comparison of | |
|---|---|---|---|---|---|---|---|
| | SNPs ID | | | Haplotype | | haplotype | |
| | Risk allele | | | frequency | | frequency | |
| Haplotype | rs490556 T | rs11748327 C | rs521660 G | Case | Control | χ² | P value |
| Haplotype1 | T | C | G | 0.727 | 0.688 | 30.7 | 3.0 × 10⁻⁸ |
| Haplotype2 | C | T | A | 0.176 | 0.213 | 37.1 | 1.1 × 10⁻⁹ |
| Haplotype3 | C | C | A | 0.053 | 0.058 | 1.4 | 2.3 × 10⁻¹ |
| Haplotype4 | T | T | A | 0.031 | 0.030 | 0.3 | 6.0 × 10⁻¹ |

When influence of age, gender, and a risk factor such as diabetes, hypertension, smoking, hyperlipidemia, or the like was examined using a one-way ANOVA and a χ² test, it was found that the above SNPs did not have an association with these factors and showed association with myocardial infarction.

### <Correlation Analysis for Angina Pectoris>

Subsequently, for the above three SNPs, samples of 2773 unstable angina pectoris (angina pectoris exhibiting severe clinical symptoms) patients were analyzed and compared with control subject samples which were used in the above third and fourth screenings.

As a result, it was found, as shown in Table 4 (UA vs. Combined CO: angina pectoris vs. control), that all of the SNPs showed significant association with angina pectoris (rs490556 P=1.1×10⁻⁴, rs11748327 P=3.4×10⁻⁵, and rs521660 P=8.7×10⁻⁵).

### INDUSTRIAL APPLICABILITY

The present invention makes it possible to predict the onset of an arteriosclerotic disease such as myocardial infarction, angina pectoris, or the like with accuracy and in a simple and convenient manner. It is considered that this not only allows a patient to avoid life-threatening dangerous conditions but also contributes to development of therapeutic agents in the future and prophylaxis of the onset of the arteriosclerotic disease.

1. A method of detecting an arteriosclerotic disease comprising the steps of analyzing a single nucleotide polymorphism present on a human chromosome 5p15.3 region and associating a result of the analysis with a risk of developing said arteriosclerotic disease.

2. The method according to claim 1, wherein said single nucleotide polymorphism is a polymorphism of a nucleotide corresponding to the nucleotide at position 61 in a nucleotide sequence of SEQ ID NO: 1, SEQ ID NO: 2, or SEQ ID NO: 3, or a nucleotide in linkage disequilibrium with said nucleotide.

3. The method according to claim 1, wherein said arteriosclerotic disease is a coronary artery disease.

4. The method according to claim 3, wherein said coronary artery disease is cardiac infarction or angina pectoris.

5. A probe for detecting an arteriosclerotic disease, said probe comprising a sequence of 10 or more nucleotides including the nucleotide at position 61 in SEQ ID NO: 1, SEQ ID NO: 2, or SEQ ID NO: 3, or a complementary sequence thereof, or agenomic sequences for probes in linkage disequilibrium with said nucleotide.

6. A primer for detecting an arteriosclerotic disease, said primer being capable of amplifying a region including the nucleotide at position 61 in the nucleotide sequence of SEQ ID NO: 1, SEQ ID NO: 2, or SEQ ID NO: 3, or genomic sequences for primers in linkage disequilibrium with said nucleotide.


SEQUENCE LISTING (3 sequences)

SEQ ID NO: 1 - DNA, 121 bp, Homo sapiens
ttttctttctacacatttacagtatataatggaagcttccaacatttataacagaacagcygtgacctgctagctaaatggtatgcaagattctataaattacagtctgaaatttgtccta

SEQ ID NO: 2 - DNA, 121 bp, Homo sapiens
aggattagcagtgaaattaatatggaaaagggcaccctgctgcccacaaacccccttcctyttccccacgtgcacaactgtactcccgcacggccacgtcgcccaccccactctgcccagg

SEQ ID NO: 3 - DNA, 121 bp, Homo sapiens
tctgaaatagcatccctcgcaaatctctatctttattacttatttatttatttttcctacrgcatatgaaattattgactgacatattttctatttaattagatattacctgaatgtgaac